(19) United States
(12) Patent Application Publication
(10) Pub. No.: US 2007/0162036 A1
(43) Pub. Date: Jul. 12, 2007

(54) Device for assisting in a total knee prosthesis implantation

(76) Inventors: Patrick Schifrine, Veyrier Du Lac (FR); Christophe Fornasieri, Meylan (FR); Pascal Vie, Mont Saint Aignan (FR); Jacques Le Saout, Landeda (FR); Thierry Musset, L'Armor Plage (FR); Patrick Laurent, Puymirol (FR); Jean-Marie Trillaud, Perrignier (FR); Philippe Ducasse, Bayonne (FR); Cyril Tarquini, Sauzet (FR); Yves Girou, La Roche Sur Yon (FR); Jean-Leon Bosredon, Bordeaux (FR); Louis Lootvoet, Namur (BE); Jean Mullier, Perwez (BE); Olivier Himmer, Bouge (BE); Jean-Paul Forthomme, St Symphorien (BE); Michael Breysse, Saint-Pierre De Chandieu (FR)

Correspondence Address: YOUNG & THOMPSON, 745 SOUTH 23RD STREET, 2ND FLOOR, ARLINGTON, VA, 22202, US

(21) Appl. No.: 10/564,547

(22) PCT Filed: Jul. 15, 2004
(86) PCT No.: PCT/FR04/01861

§ 371 Date: Jun. 14, 2006

(30) Foreign Application Priority Data
Jul. 16, 2003 (FR) 03/08698

(51) Int. Cl. A61F 5/00

(52) U.S. Cl. 606/87

(57) ABSTRACT

A device for setting a total knee prosthesis includes: a tensioning component (1) having: a tibial plate (2), a slide (5) mobile along a rail (3) and elements (6, 11) for being fixed to an ancillary component including a centro-medullar rod and a tibial plate and capable of receiving variable-width wedging elements; and a motoring elements (8, 10) for tensioning the knee when the plate is applied on a tibial section and the ancillary component (20) secured to the slide, a perforating guide capable of being mounted on the rail (3) and having drilling holes; the guide capable of including or of being associated with a member for palpation of the anterior part of the femur; and locating elements (EF) for determining the interarticular space available in flexure, thereby enabling the distal femoral section plane or the posterior section plane to be accurately determined.

The present invention relates to an auxiliary device for a total knee prosthesis, which device is intended to bring about the tensioning of the articulation and facilitate the cutting and balancing operations for the practitioner during the operation.

The invention also relates to surgical procedures for positioning a total knee prosthesis using this device.

A number of auxiliary devices are already known which are intended to guide the surgeon during the positioning of a total knee prosthesis and, in particular, to help him determine the cutting planes. It should be noted in this regard that the positioning of a prosthesis requires, in addition to the tibial cut in the region of the plate for receiving the tibial component of the prosthesis, a distal cut of the femur which is supported on the femoral component of the prosthesis and a posterior cut of the femur in the region of the condyles and, if necessary, other cuts, including an anterior cut.

Auxiliary devices are thus described in EP-A-0327249.

An improved device is described in document WO98/25526, this device comprising an ancillary component which has a tibial plate followed by a centro-medullary rod which is intended to be introduced into the femoral medullary canal, this plate being capable of receiving adjusting means of variable thicknesses which are intended to be supported on the tibial plate after the tibial cut in order, on the one hand, to allow the laxity of the articulation of the knee in the state of extension to be measured and balanced and, on the other hand, to determine the location of the posterior femoral cut, in the state of flexion, in order to provide an articulation balance with a ligament tension which is suitable in all positions of the knee from extension to complete flexion.

The adjusting means, of variable thicknesses, are such that the total thickness of the plate and the shim is equal to the distance between the proximal tibial cutting plane and the distal end of the femur shortest after ligament balance has been obtained by means of distraction of the articulation between the plate and the distal end of the femur.

The contents of the application WO98/25526 and the corresponding U.S. application Ser. No. 3,19,709 are incorporated herein by reference.

The present invention proposes to provide a tensioning device for a total knee prosthesis which allows the knee to be placed in a state of ligament tension in natural positions in a simple manner and optionally allows the position of the distal femoral cutting plane and the position of the posterior femoral cutting plane and the anterior femoral cutting plane to be determined, in a manner which is very simple and practical for the surgeon.

Another object of the invention is to allow, at the same time, the size of the femoral prosthesis which has to be positioned to be estimated in an optimum manner.

Another object of the invention is to provide a device of this type which allows cutting guides to be positioned in an optimum manner at the distal end of the femur, whilst being able to carry out the above-mentioned functions if the surgeon prefers to begin by proceeding immediately with a distal femoral cut.

The overall object of the device is to allow cuts to be carried out in accordance with the prosthesis selected in order to provide a ligament balance of very high quality over the entire flexion path of the knee.

The invention relates to a device for positioning a total knee prosthesis comprising:

a tensioning component having:
a plate which is capable of being supported on a tibial cutting surface,
a slide which is capable of being displaced on a sliding means in a direction substantially perpendicular relative to the plate and which has means for being fixedly joined temporarily to an ancillary component which comprises a centro-medullary rod and a tibial plate and which is capable of receiving adjusting means of variable thicknesses, positioned beforehand at the end of the femur when the knee is in a state of flexion at approximately 90°,
and a motor means, such as, for example, a screw and nut type system, which allows the surgeon to displace the slide and tension the knee when the plate is pressed on the tibial cut and the ancillary component which is fixedly joined to the slide,
a drilling guide which is capable of being mounted on the sliding means on the slide and which has drilled holes which allow the subsequent positioning on the femur of a cutting block which in particular allows the posterior femoral cut to be brought about,
the guide preferably being able to comprise or be associated with a means for palpating the anterior portion of the femur in order to position the guide in alignment with this anterior portion,
and reference means which determine the position of the slide and/or the guide relative to the plate of the tensioning component and therefore determine the interarticular space available in the state of flexion.

The device thus allows either the position of the distal femoral cutting plane to be determined by determining the difference between the spacing in the state of extension and the space in the state of flexion, or, in the case of a distal cut which is carried out immediately, the position of the posterior femoral cutting plane to be determined in order to obtain approximate equality between the spacing in the state of extension and the space in the state of flexion.

Advantageously, the drilling guide may be in the form of a set of several guides of various dimensions, which allows the surgeon to adapt a guide which corresponds to the size of the femur in order to be able to determine an optimum posterior cutting plane, and in particular does not involve any significant bone resection.

The device according to the invention may further comprise a size estimation component which is capable of being mounted on the sliding means in order to be able to estimate, using a reference means, the size of the femur and allow the correct drill guide component to be selected. To this end, the dimension of the plate may advantageously be such that the femoral end can be received between the plate and the component, in the manner of a calliper rule.

The device may further comprise a distal cutting guide support which has a member which is capable of sliding on the sliding means and from which an arm extends which will extend parallel with the axis of the knee in a state of flexion, and which has means, for example, a catch mechanism, and/or a location graduation, for receiving and fixing the distal cutting guide at a precise location, the precise location preferably being determined by the calculation of the difference between the space in the state of flexion and the spacing in the state of extension.

The device according to the invention may in particular be in the form of a kit comprising the tensioning component, the drilling guide and, optionally, the component for estimating the size of the femur.

This set may advantageously be supplemented by the ancillary component or a set of components of this type, with adjusting means in order to determine the articular spacing in the state of extension.

This spacer device in the state of extension is advantageously described in the applications WO98/25526 and US 319709 which are incorporated herein by reference.

The ancillary component which comprises a centro-medullar rod and a tibial plate and which is capable of receiving a shim advantageously has means which allow it both to be positioned in a precise manner on the slide and to be temporarily fixed thereto. These means may, for example, comprise positioning notches and an aperture which allows the passage of a fixing, for example, a bayonet type fixing which clamps the plate of the ancillary component against the slide.

In a particular embodiment of the tensioning component, it comprises, extending from the plate, sliding means, on or in which the slide is capable of sliding, this slide preferably being able to be displaced by means of a screw which is axially fixed but which is able to rotate relative to the sliding means and which co-operates with a corresponding threaded portion or nut of the slide in order to allow the surgeon, by actuating the end of the screw remote from the plate, to slide the slide and place the knee in a state of tension.

Preferably, the sliding means have an internal runner in which the slide is guided, so that the outer surface of the sliding means allows the drilling guide and other components of the device to be guided, the slide having a portion, for example, a relief, which allows the drilling guide to be moved on the device.

The drilling guide is preferably constructed so as to receive a palpating arm which is capable of pressing on the anterior surface of the femoral end in order to limit the insertion of the guide on the guiding means in order to determine the drilling position.

In a variant, the selection of a drilling guide having suitable dimensions and the precise positioning thereof on the slide in a state of tension may carry out this function of locating the drilling.

Advantageously, the slide also has reliefs which allow precise positioning, relative to the slide, of the plate of the ancillary component and a rapid fixing means, for example, of the bayonet type, which allows the rod to be temporarily fixedly joined relative to the slide in the position in which the plate of the rod has been received on the slide.

Advantageously, the size estimation component for palpating the anterior end of the femur may be formed in one piece with a member which is capable of sliding on or in the sliding means, this sliding member having a transverse palpating arm which is preferably articulated about a shaft parallel with the sliding axis.

The palpating arm may comprise, if necessary, a rod which can be displaced both longitudinally and transversely relative to the arm.

The invention also relates to procedures for positioning a total knee prosthesis using the device according to the invention.

In one method of implementation, a procedure of this type may comprise the following steps:
after resection of the proximal articular end by means of a tibial cut in the region of the tibial plate, positioning an ancillary component, which comprises a centro-medullar rod and a tibial plate and which is capable of receiving a shim, in the femoral medullar canal after the knee has been placed in a state of flexion so that the plate of the ancillary component is pressed against the distal face of the femoral condyles,
after being placed in an extended state again, balancing the knee in the state of extension, as described, for example, in document WO98/25526 and measuring the spacing in the state of extension, for example, using the shims described in that document; this determines a measurement which is known as the spacing in the state of extension,
after the knee has been placed in a state of flexion, positioning the tensioning component by fixing the slide against the plate of the ancillary component then, optionally, estimating the size of the femur using the size estimation component which can be mounted in a sliding manner on the tensioning component, the femur in a state of flexion being received between the plate of the tensioning component and the palpating means of the support which is mounted on the tensioning component,
after removing this estimating component, if applicable, placing the knee in a state of ligament tension in a flexion position, the plate of the tensioning component being supported on the tibial cutting plate and the means for displacing the slide being activated until a suitable tension is obtained, the femur being able to turn freely in terms of rotation about the centro-medullar rod of the ancillary component during the tensioning displacement for correct ligament balance,
positioning the drilling guide of the size previously estimated,
confirming the size using the anterior palpating arm and reading the space in a state of virtual flexion, for example, by reading a graduation on the tensioning component which is, for example, graduated on the runner thereof, in order to control the position of the drilling guide on the tensioning device,
drilling two cutting guide holes, after the optimisation of the anteroposterior balance has been carried out,
positioning a distal cutting guide, for example, by means of screwing on the anterior face of the femur, then adjusting the distal cutting plane after calculation of the distal cutting adjustment distance equal to the difference between the space in the state of flexion and the spacing in the state of extension, then definitively fixing the distal cutting guide on the anterior portion of the femur using spindles,
removing the extension component then the ancillary component,
carrying out the distal cut using the distal cutting guide, then removing the distal cutting guide,
positioning a cutting block using holes which are produced using the drill guiding component then carrying out the various other cuts using this cutting block, that is to say, the posterior cut, the anterior cut and the two associated chamfers.

In another method for carrying out a procedure according to the invention, the surgeon begins by positioning the distal cutting guide and manually adjusts the position of the distal cut; after fixing the distal cutting guide by means of spindles or screws, he carries out the distal cut;
he then verifies the spacing in the state of extension using conventional adjusting or extension means;
then positioning the ancillary component as in the preceding implementation method;
positioning the tensioning component on the plate of the rod and optionally estimating the size of the femur in accordance with the preceding implementation method,
placing the bent knee in a state of ligament tension which brings about the optional rotation of the femur in order for it to be adapted to the correct ligament tension, then locking the tensioning device in the tension position thereof,
positioning the drilling guide of the size previously estimated and confirming or modifying this size,
reading the virtual space in a state of flexion on the tensioning device, these operations being similar or identical to those of the corresponding step of the preceding method;
positioning the drilling guide so that the space in the state of flexion reproduces, preferably in an identical manner, the spacing in the state of extension imposed by the initial distal femoral cut,
drilling two holes which are intended to fix the cutting guide,
removing the drilling guide, the tensioning device, then the ancillary component,
positioning the cutting block in the holes which have been produced using the drilling guide, then carrying out the various cuts in accordance with the final step of the preceding method.

Other advantages and features of the invention will be appreciated from a reading of the following description, given by way of non-limiting example and with reference to the appended drawings, in which:

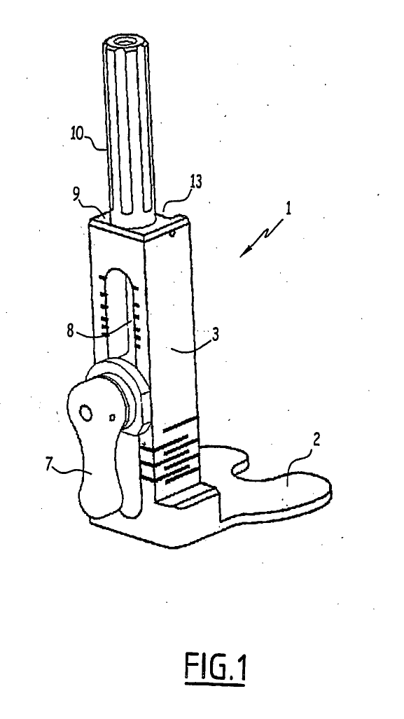

FIG. 1 is a perspective view of the tensioning component,

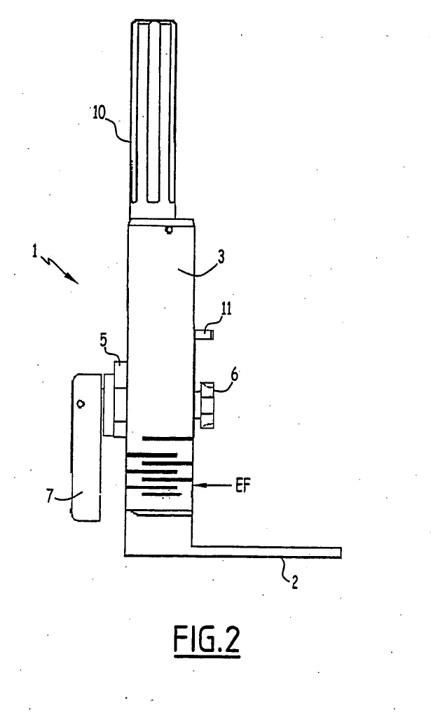

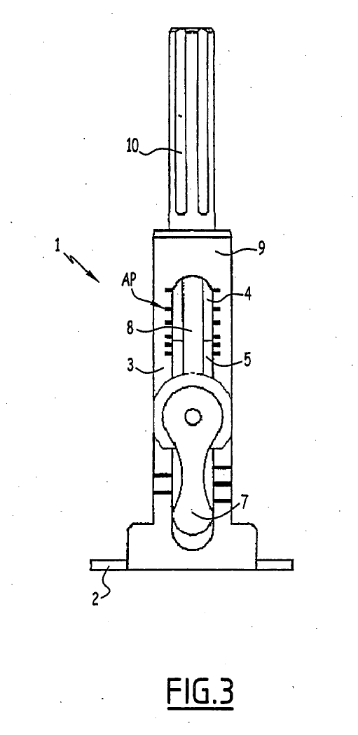

FIGS. 2, 3 and 4 are front, side and plan views of this device, respectively,

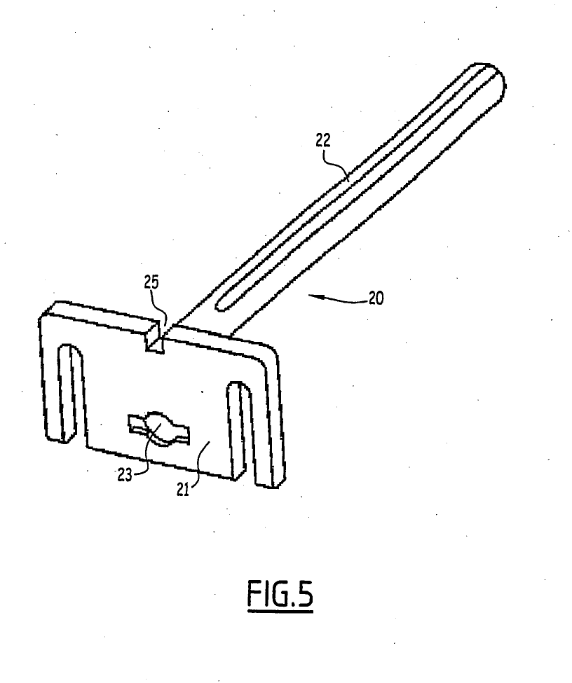

FIG. 5 is a perspective view of an ancillary component which comprises a centro-medullar rod and a tibial plate and which is capable of receiving a shim,

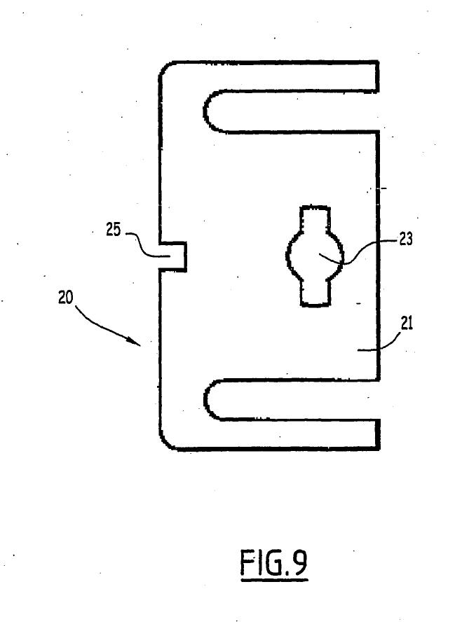

FIGS. 6 to 9 are front, side, upper and lower views of this rod, respectively,

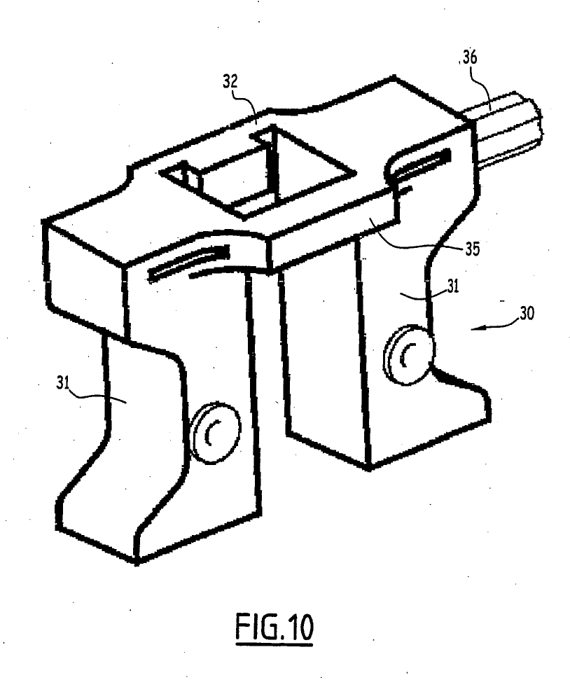

FIG. 10 is a perspective view of a drill jig,

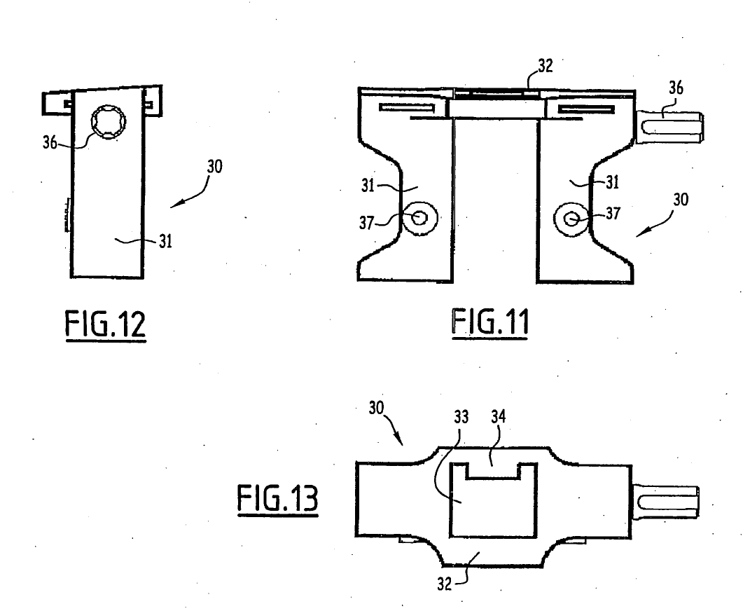

FIGS. 11, 12 and 13 are front, side and plan views of this drill guiding component,

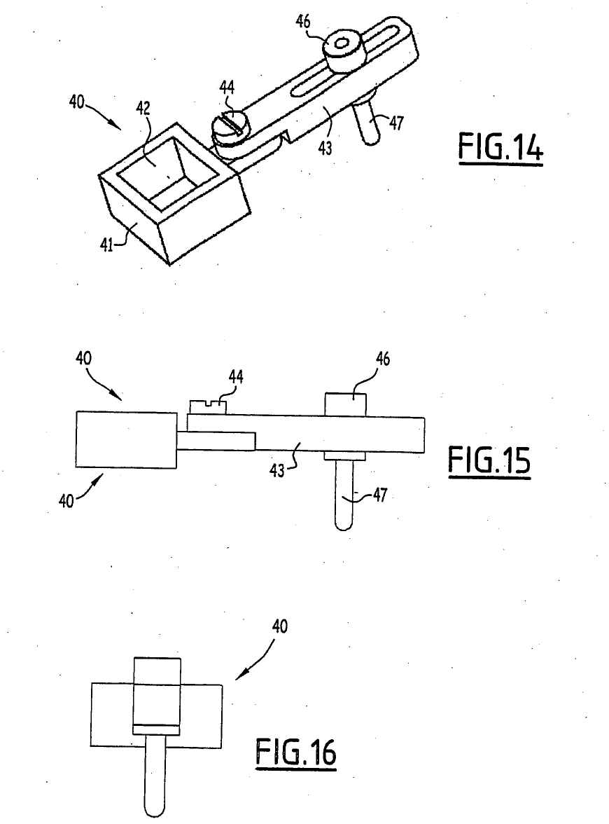

FIG. 14 is a perspective view of a size estimation component,

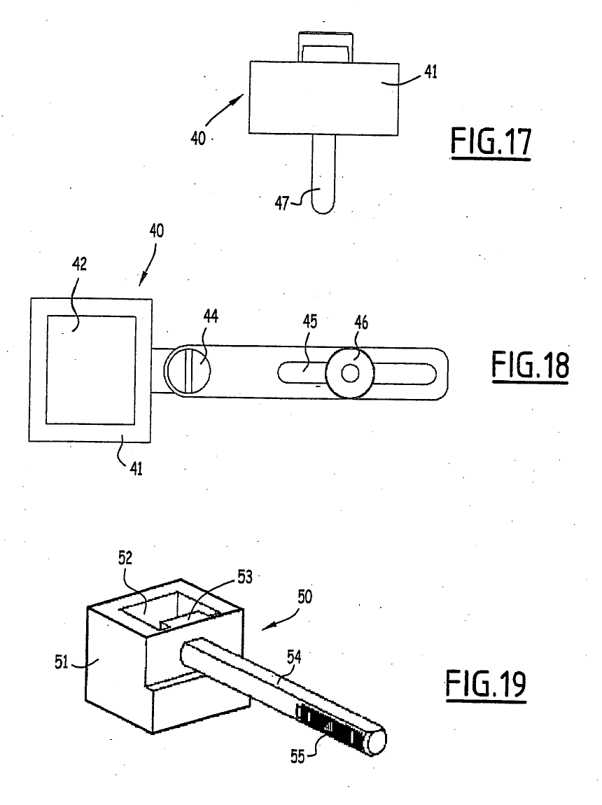

FIGS. 15, 16, 17 and 18 are front, side and plan views of this component, respectively,

FIG. 19 is a perspective view of a distal cutting guide support,

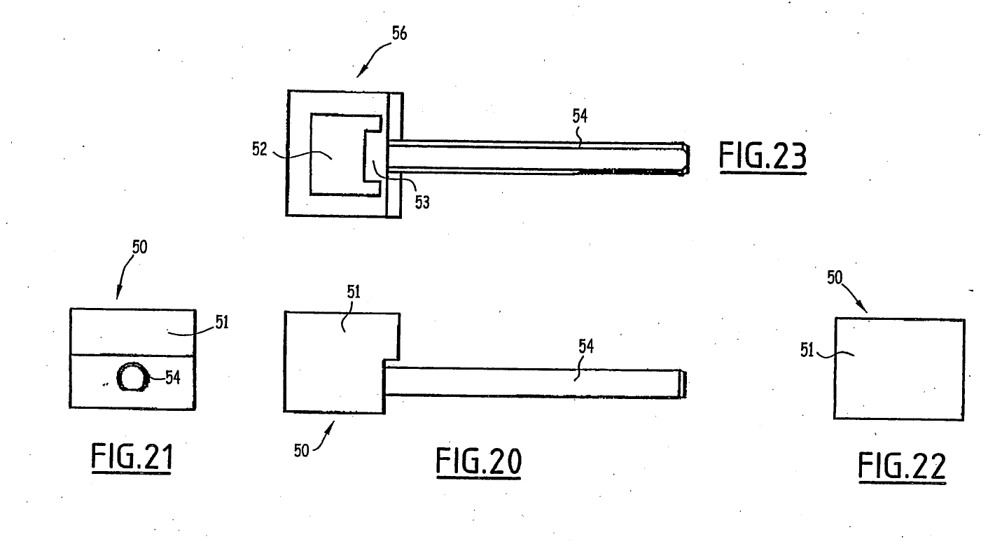

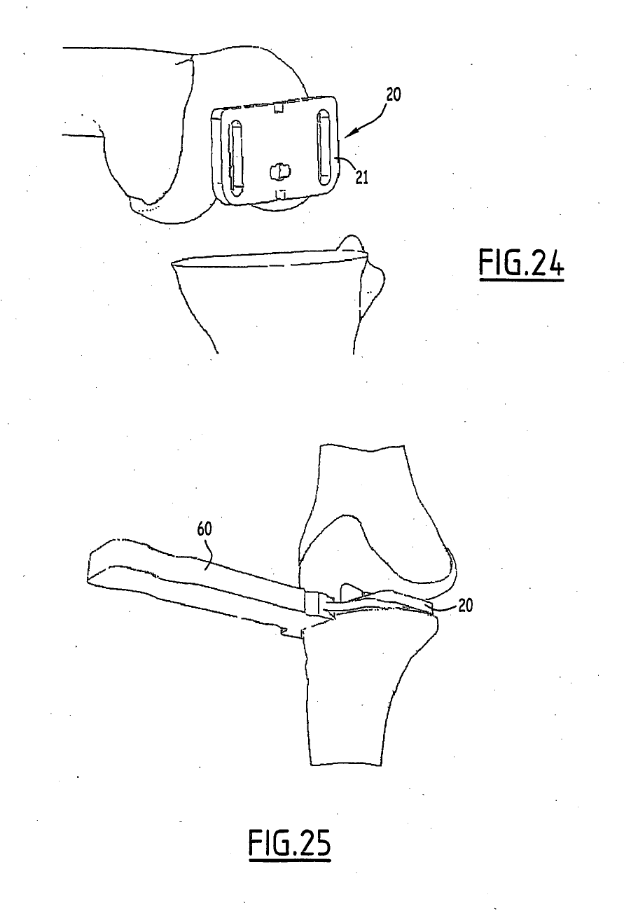

FIGS. 20 to 23 are front, side and plan views of this component, respectively,

FIGS. 24 to 32 are schematic perspective views of various stages of an operation for positioning a knee prosthesis using the device according to the invention,

FIGS. 33 to 41 are views of the various stages of an operation of this type for positioning a prosthesis in another implementation method of the invention.

The device according to the invention comprises a knee extension device 1. This comprises, starting from a plate 2, a double upright 3 which is perpendicular relative to the plate and which defines an inner aperture 4 in the same direction, and whose faces form a sliding means for guiding a slide 5 in translation.

The slide 5 has a bayonet fixing means 6 at the side of the upright 3 facing the plate 2, this bayonet fixing means being activated by means of a rod which extends through the slide, a lever 7 allowing the bayonet fixing means 6 to be rotated. At the upper face thereof, the slide 5 continues in one piece as a threaded rod 8 which freely extends through the upper end 9 of the upright and is introduced into the inner threaded portion of a nut 10 which is mounted so as to be free in terms of rotation but fixed in terms of translation on the end 9 of the upright; the nut 10 having a form which is elongate upwards with grooves in order to form an activation wheel. Finally, the upper end of the slide 5, at the side facing the base 2, has a transverse pin 11 which protrudes from the upright 3.

It will therefore be appreciated that, by turning the wheel-like nut 10 in one direction or the other, the threaded rod 8 is raised or lowered relative to the upright 3, and consequently the slide 5 which is guided in the internal sliding means formed by the upright 3. Consequently, if a component is fixed against the slide by means of the bayonet fixing means 6, or if a component is resting on the pin 11, this component will follow the movements of the slide.

The device illustrated is intended to co-operate with an ancillary component according to the applications WO98/25526 and US 319709 and this matter will be referred to in FIGS. 5 to 9.

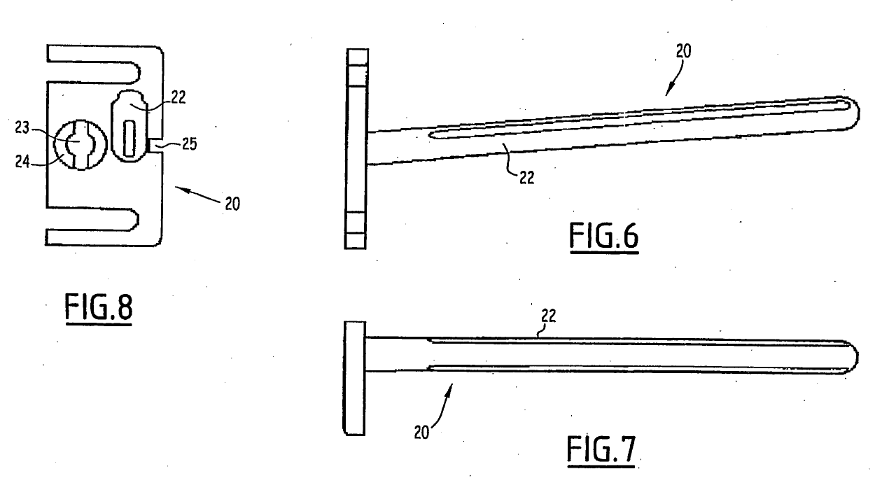

This component comprises a base or plate 21 which has, on one of the faces thereof, and facing an anterior edge from an anatomical point of view for the femur, an actual centro-medullar rod 22 which, in a sagittal plane, as illustrated in FIG. 7, is substantially perpendicular relative to the base 21 but which, in a frontal plane, as illustrated in FIG. 6, has an inclination relative to the normal to the base corresponding to the normal femoral valgus.

This ancillary component is intended to allow a ligament balance in the state of extension to be sought by the adjusting means and intended for the optional use of distraction means in the state of extension.

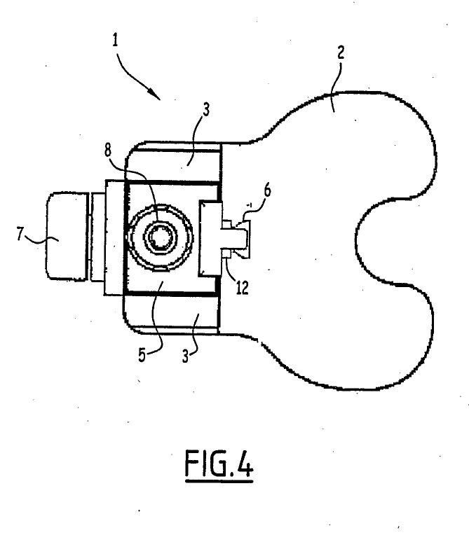

It can be seen that, in alignment in a sagittal plane with the lower end of the rod 22, the base 21 has an oblong passage 23 which has a central hole which continues at one side and the other by means of diametrically opposed notches which are intended to allow the passage of the bayonet head 6. This shape is produced at the bottom, arranged at the lower side of the base, of a circular countersinking 24 in such a manner that it will be appreciated that, when the fixing head 6 is introduced, via the lower side which can be seen in FIG. 5, into the passage 23, a washer 12 located on the slide below the bayonet head 6 is inserted into the circular countersinking 24 for precise positioning of the shape of the slide, and therefore the component 1, relative to the component 20. The bayonet head 6, which projects beyond the upper end of the base 21, and which is in the form of a ramp, as can be seen in FIG. 4, definitively locks the base 21 against the slide, whilst the pin 11 is inserted into a corresponding notch 25 of the base 21 in order to complete the reciprocal shape locking of the two components.

With the ancillary component 20 being locked in this manner on the slide, it is possible to position a drill jig or guide and reference will be made to this matter in FIGS. 10 to 13.

This jig is generally U-shaped having two legs 31 and a base 32, this base 32 having a rectangular central passage 33 which extends the empty space between the two legs 31 and which has an inner relief 34.

The drill jig can thus be mounted so as to slide on the upright 3 which is used as an external sliding means for the two legs 31 and the hole 33, precise positioning further being provided by the introduction of the relief 34 via the indentation 13 which is formed in the upright at the side facing the base 2.

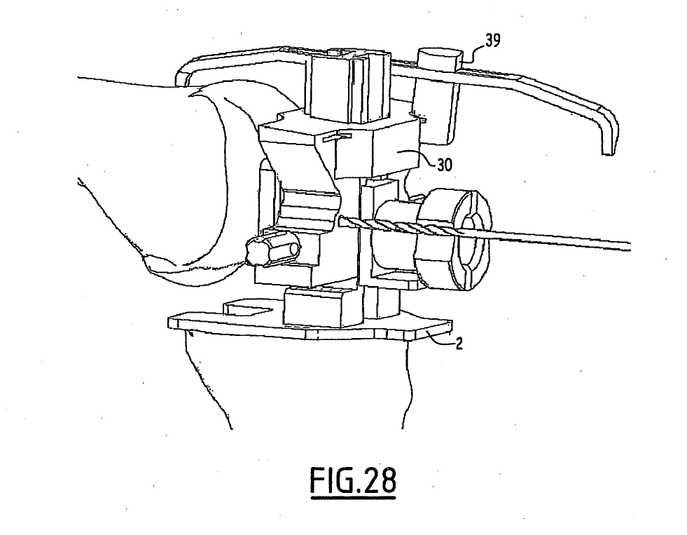

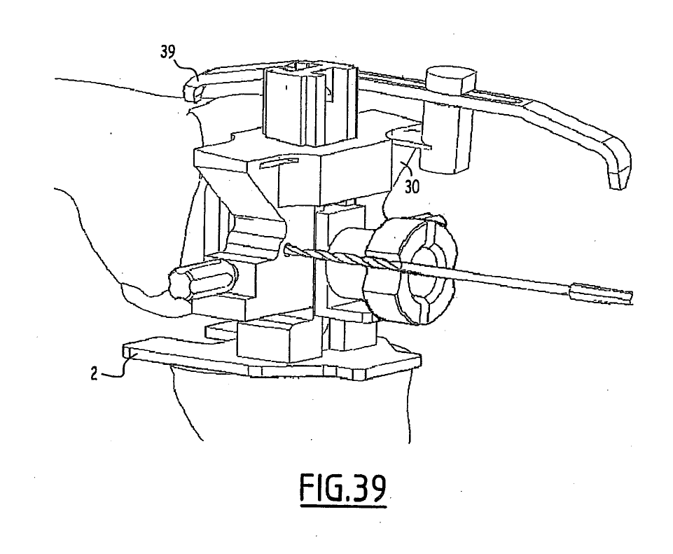

The jig also has apertures 38 which allow the positioning of a palpating arm 39, which can be seen, for example, in FIGS. 28 and 39, and which allows the position of the jig 30 to be determined in a precise manner relative to the anterior edge of the femur.

As can be seen in FIG. 12 in particular, the upper surface of the drill jig 30 is inclined in accordance with a plane which is directed towards the rod 22 when the drill jig 30 is positioned in such a manner that an edge 35 of the drill jig is pressed against the edge of the base 21 which carries the notch 25 when it is also in position on the slide of the device, this inclined plane being intended to indicate the anterior femoral cutting plane.

A screw fixing means 36 allows the drilling guide to be fixed relative to the upright 3, the end (not illustrated) of the screw 36 being pressed against this upright.

Two holes 37, which extend through the legs 31, act as a guide for drilling spindles in order to drill, in the end of the femur, the fixing holes for a conventional cutting guide.

The lower faces of the legs 31 move in relation to an EF graduation carried by the runner 3 in order to form the reading index for the space in a state of flexion on this graduation.

The tensioning device 1 may further receive a component 40 for estimating the size of the femur, which is described with reference to FIGS. 14 to 18.

This device has a square member 41 which is provided with a square central passage 42 which allows precise running on the outer surfaces of the upright 3. At the side directed towards the base 2, this member has an arm 43 which preferably has an articulation 44, the longest portion of this arm 43 itself forming a runner 45 in which the support 46 can slide, from a palpating point 47, and it will therefore be appreciated that it is thus possible to arrange the palpating point 47 at the location in which the femur has the maximum anterior dimension thereof.

By means of this palpating arm, it is possible to use the assembly formed by the tensioning device 1 and the component 40 which is mounted so as to slide on the upright 3 as a calliper rule when the plate 2 is pressed against the posterior portion of the end of the femur, whilst the palpator 47 is pressed against the anterior portion of this femoral end. The anteroposterior dimension of the femoral end can thus be determined by reading the position of the lower edge 48 of the member 41 on the AP graduation which can be seen on the upright 3, in particular FIG. 5, at the side having the lever 7.

The device according to the invention may further advantageously comprise a distal cutting guide support 50 which can be seen in FIGS. 19 to 23. The support comprises a member 51 which is provided with a square passage 52 and which has a relief 53 which is intended to co-operate with the indented portion 13 of the upright 3 so that the sliding of the member 51 on the upright 3 is carried out with great precision. At the side of this relief 53 and therefore at the side of the base 2, this member has a perpendicular rod 54 on which it is possible to fit a distal cutting guide which can thus be positioned correctly at the anterior side of the femoral end at the desired distance from the upright 3, and therefore the reference plane determined by the proximal face of the plate 21 of the ancillary component 20.

To this end, the arm 54 has a catch portion 55 which allows complete fixing of the distal cutting guide on the arm 54, the arm having a graduation (not illustrated) which is referenced relative to the proximal face of the plate 21 in order to position the distal cutting guide at the distance equal to the difference between the space in the state of flexion and the spacing in the state of extension.

A method using the device will now be described with reference to FIGS. 24 to 32.

The steps of this method are as follows:

1. (FIG. 24) positioning the ancillary component 20, the rod 22 being inserted into the medullar canal and the base being pressed by means of the upper face thereof against the lower end of the femoral condyles; this operation is carried out with the knee in a bent state.

2. (FIG. 25) after the knee has been placed in the extended state again, the ligaments are tensioned by moving the tibial cutting plate away from the femoral end using a distraction device 60, such as the known adjusting means, in order to obtain a rectangular tibiofemoral extension spacing in which the axes of the tibia and the femur are correctly positioned, this spacing in the state of extension then being read by the surgeon in conventional manner;

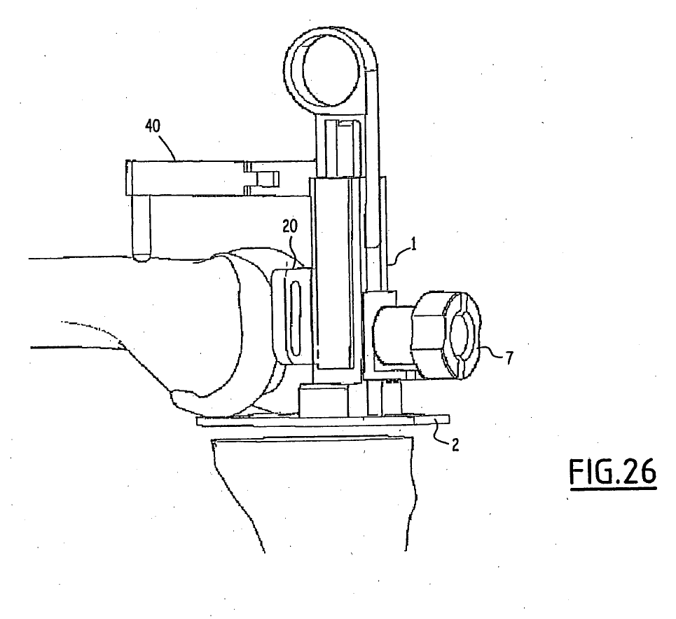

3. (FIG. 26) after having positioned the knee in a state of flexion again, the device 1 is fixed, as illustrated in FIG. 26, against the lower face of the base 21 using the bayonet fixing means and the button or wheel 10 is rotated until the plate 2 is brought into contact with the posterior end of the femoral condyles. It is then possible to position the size estimation device 40 on the upright 3 and read on the AP graduation the anteroposterior dimension of this femoral end in the manner of a calliper rule; in FIG. 26, the device 40 has mounting and reference means which form a variant of those illustrated in FIG. 14.

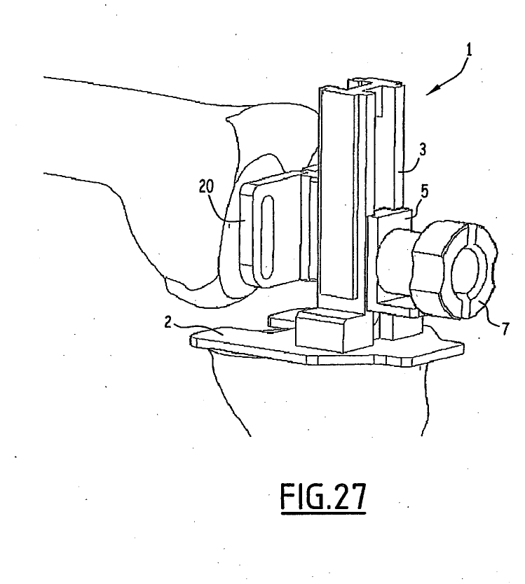

4. after the component 40 has been removed, and as can be seen in FIG. 27, the knee is placed in a state of ligament tension by rotating the lever 7, the base 2 being supported on the tibial cutting plate. The femur can thus rotate freely about the rod 22 located in the medullar canal thereof and assume the correct position until the degree of ligament tension desired by the surgeon is achieved;

5. (FIG. 28) with the device being locked in this position, the drill jig 30 provided with the palpating arm 39 is positioned on the upright 3 in order to confirm the anteroposterior size of the end of the femur. If necessary, the drill jig is replaced with another jig of a different size in order to optimise the dimension and the space in the state of flexion read on the EF graduation; when the jig is considered to be in the correct position, two drill spindles are passed through the two holes 37 thereof and the holes which will act as a guide for positioning the cutting guide are drilled in the end of the femoral condyles; at this time, the surgeon reads the space in the state of flexion by examining the position of the lower end of the jig 30 on the EF scale which is fixed to the upright 3;

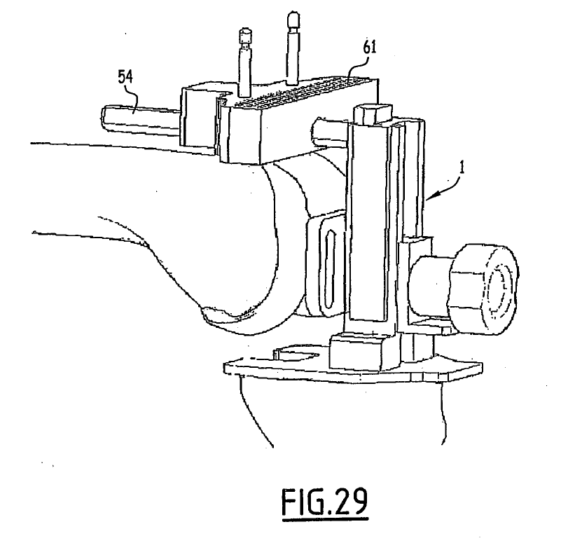

6. (FIG. 29) after having noted this space, the drill jig is removed and the distal cutting guide support 50 is positioned on the upright 3, on the arm of which guide support the distal cutting guide 61 is fitted. This distal cutting guide 61 is placed on this arm at a distance equal to the difference between the space in the state of flexion which had been read on the EF graduation and the spacing in the state of extension which had initially been read in phase 2. With the jig being held in this position along the arm 54, the surgeon fixes the jig by means of spindles which are screwed in the anterior portion of the femoral end;

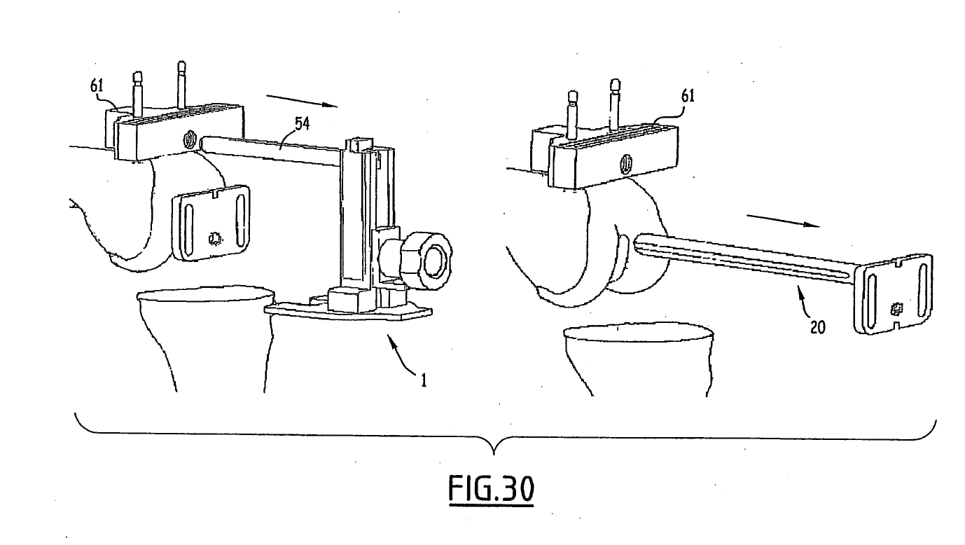

7. (FIG. 30) the assembly comprising the tensioning component 1 and the cutting guide support 50 is then removed, then the ancillary component 20 is extracted;

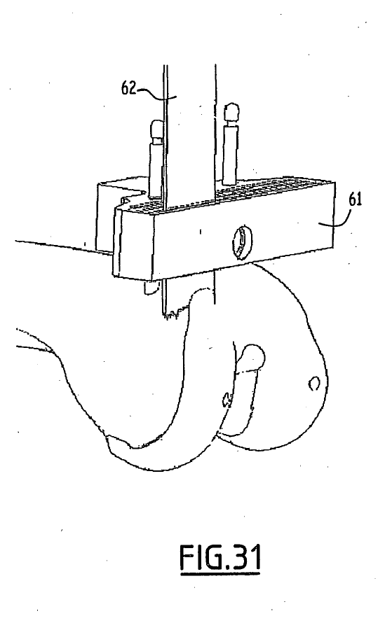

8. (FIG. 31) it is then possible to carry out the distal cut in conventional manner in the position which has been defined by the distal cutting guide 61 which guides the cutting tool 62;

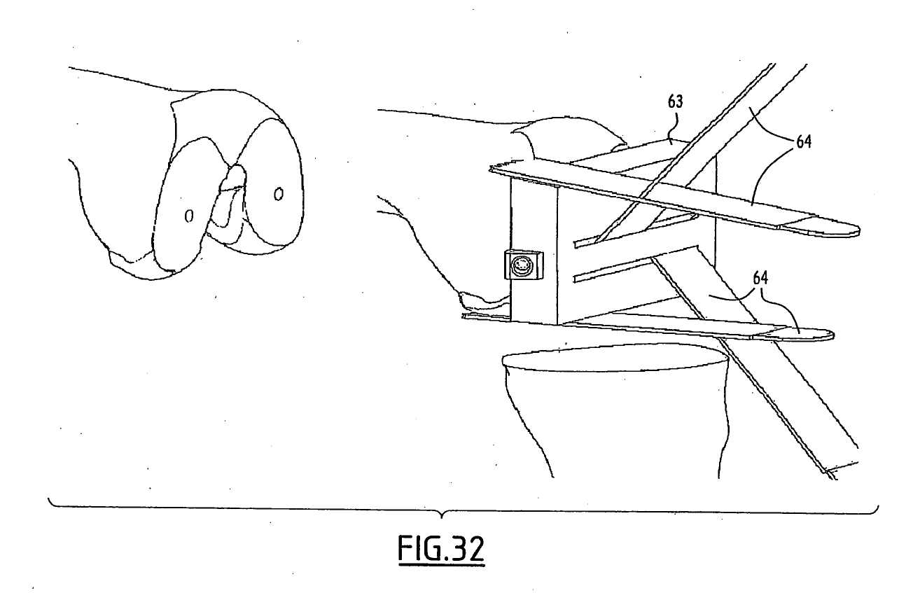

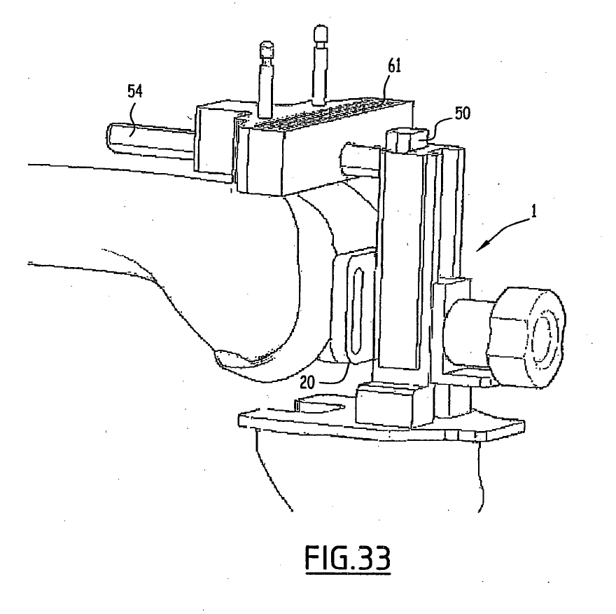

9. after the distal cutting guide 61 has been removed, the cutting block 63 is positioned in the holes produced during phase 5, until the cutting block is pressed and fixed against the distal cut of the femur carried out during step 8 (FIG. 32). The surgeon can then carry out the various other cuts using the tools 64, and in particular the posterior cut, the cutting guide being constructed, in terms of the dimensions thereof relative to the locations determined by the holes, in order to remove the optimum quantity from the posterior femoral edge. He may optionally also carry out the anterior cuts and chamfer cuts of the femoral end, if necessary.

The surgeon can then continue the procedure, after the cutting blocks have been removed, and position the components of the complete knee prosthesis.

The device according to the invention can also be used in a variant of the procedure in which, as is often the case, the surgeon initially carries out the distal cut, as described in FIGS. 33 to 41. This procedure may comprise the following steps:

1. on the femur in a state of flexion in which he has introduced the rod of the ancillary component 20, then fixed the component 1, in a position in which the base thereof is supported on the tibial cutting plate, the surgeon places the distal cutting guide support 50, then positions thereon, at the location which he considers optimum, the distal cutting guide which he fixes to the anterior edge of the femur by means of spindles (FIG. 33);

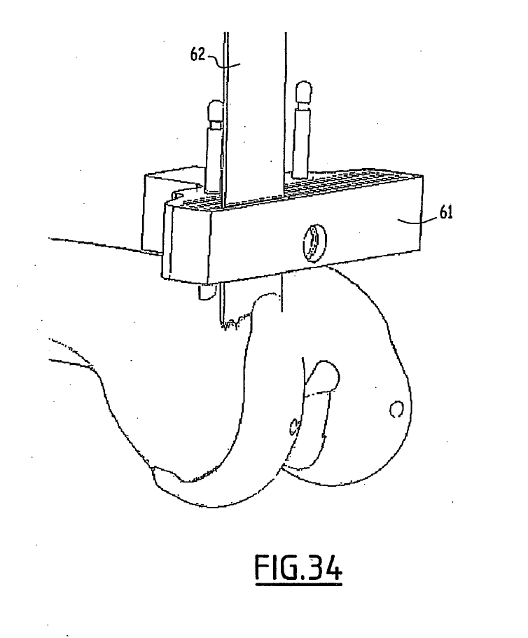

2. after the components 1, 20 and 50 have been withdrawn, he carries out the distal cut (FIG. 34);

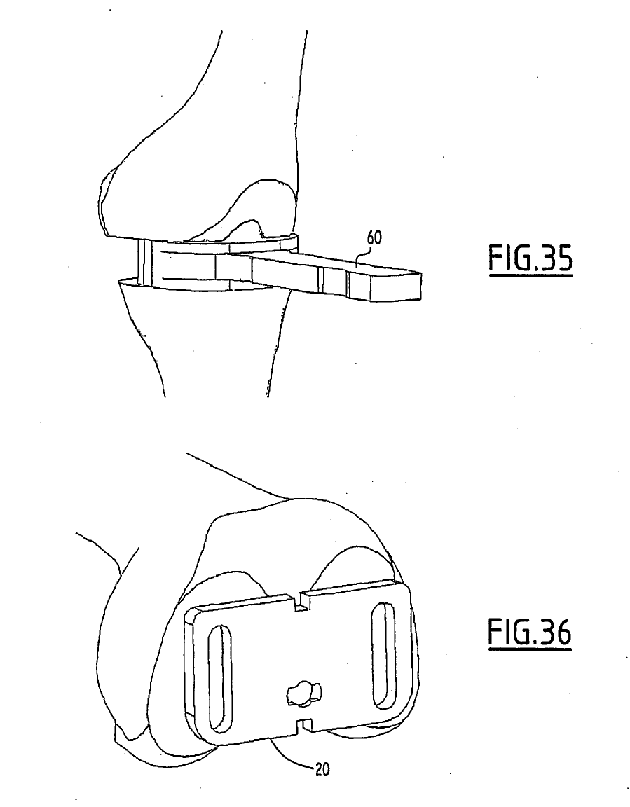

3. after having placed the knee in a state of extension again, as illustrated in FIG. 35, he inspects the spacing in the state of extension between the two tibial and femoral planes produced in this manner using conventional means 60;

4. after having placed the knee in a state of flexion again, he positions the ancillary component according to FIG. 36;

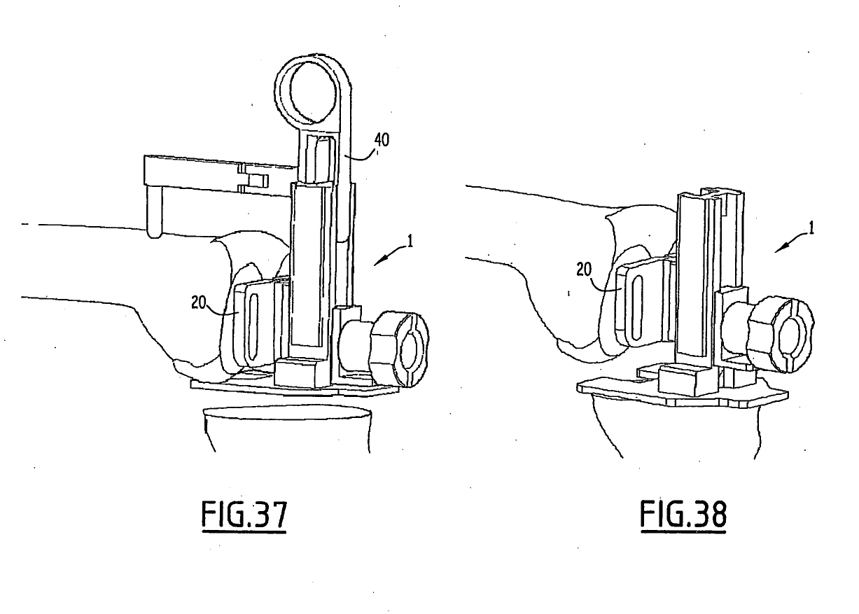

5. as can be seen in FIG. 37, he repositions the tensioning component 1 and, using the component 40, measures the anteroposterior dimension of the femur in the manner of a calliper rule;

6. after the component 50 has been removed, as can be seen in FIG. 38, he places the ligaments in a state of tension which brings about the adaptation of the femur by means of rotation about the rod 22, until the tensioning component 1 is locked in a state of tension;

7. then positioning the drilling guide 11, confirming the femoral size using a palpating arm which this component is provided with as illustrated in FIG. 39; after confirming the femoral size and, if necessary, replacing the drill jig with another of a different size, seeking a position in which the space in the state of flexion on the EF graduation is equal to the spacing in the state of extension, then drilling the holes for fixing the cutting block guide by means of spindles which are screwed through the holes 37; it is then possible to verify, on the EF graduation, the space in the state of flexion obtained on the EF scale;

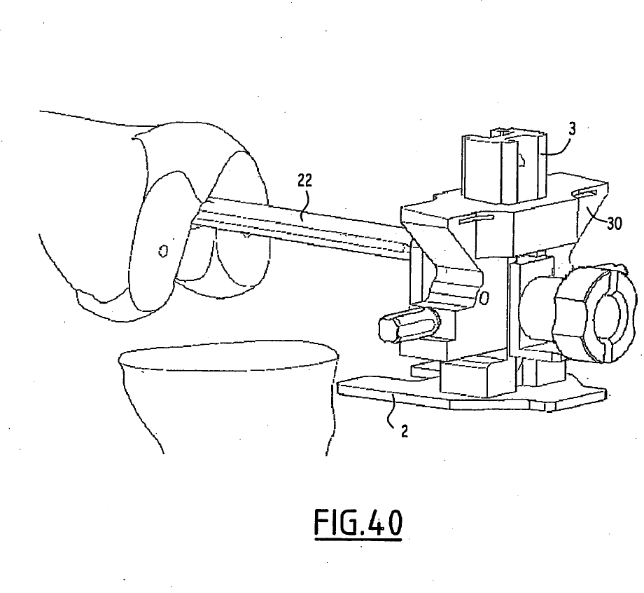

8. as can be seen in FIG. 40, he then removes all the components which have been fixed;

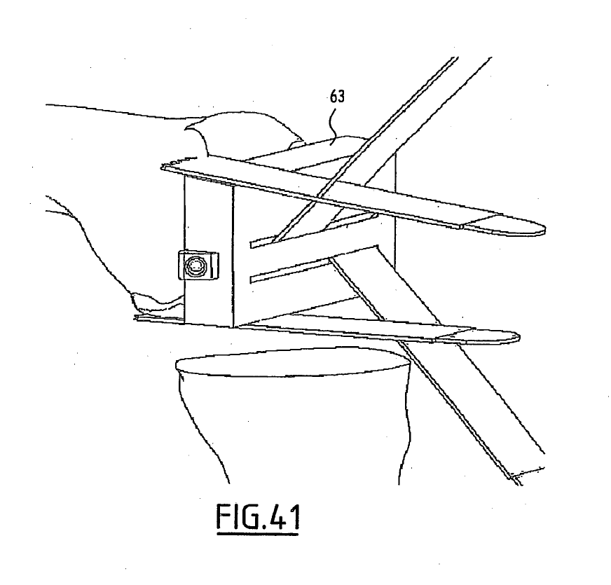

9. he can then, using the holes which have just been produced, position the cutting block as illustrated in FIG. 41 and carry out the various cuts, including the cut of the posterior edge of the femoral end.

1. Device for positioning a total knee prosthesis comprising:
a tensioning component (1) having:
a plate (2) which is capable of being supported on a tibial cutting surface,
a slide (5) which is capable of being displaced on a sliding means (3) in a direction substantially perpendicular relative to the plate (2) and which has means (6, 11) for being temporarily fixedly joined to an ancillary component (20) which comprises a centro-medullary rod and a tibial plate and which is capable of receiving adjusting means of variable thicknesses, positioned beforehand at the end of the femur when the knee is in a state of flexion at approximately 90° allowing spacing in an extended state of the articulation to be obtained,
and a motor means (8, 10) which allows the surgeon to displace the slide (5) and tension the knee when the plate is pressed on the tibial cut and the ancillary component (20) which is fixedly joined to the slide,
a drilling guide (30) which is capable of being mounted on the sliding means (3) and which has drilled holes (37) which allow the subsequent positioning on the femur of a cutting block which allows the posterior femoral cuts to be brought about,
the guide (30) being able to comprise or be associated with a means for palpating the anterior portion of the femur in order to position the guide in alignment with this anterior portion,
and reference means (EF) which determine the position of the slide (5) and/or the guide (30) relative to the plate (2) of the tensioning component (1) and therefore determine the interarticular space available in a state of flexion, the device thus allowing either the position of the distal femoral cutting plane to be determined by determining the difference between the spacing in the state of extension and the space in the state of flexion, or, in the case of a distal cut which is carried out immediately, the position of the posterior femoral cutting plane to be determined in order to obtain approximate equality between the spacing in the state of extension and the space in the state of flexion.

2. Device according to claim 1, characterised in that it comprises a size estimation component (40) which is capable of being mounted on the sliding means (3) in order to be able to estimate, using a reference means, the size of the femur and allow the correct drill guide component (30) to be selected.

3. Device according to claim 2, characterised in that the dimension of the plate (2) is such that the femoral end can be received between the plate and the component (40), in the manner of a calliper rule.

4. Device according to claim 1, characterised in that it comprises a distal cutting guide support (50) which has a member (51) which is capable of sliding on the sliding means (3) and from which an arm (54) extends which will extend parallel with the axis of the knee in a state of flexion, and which has means for receiving and fixing the distal cutting guide at a precise location, the precise location being able to be determined by the calculation of the difference between the space in the state of flexion and the spacing in the state of extension.

5. Device according to claim 1, characterised in that the tensioning component (1) comprises, extending from the plate (2), sliding means (3), on or in which the slide (5) is capable of sliding, this slide (5) being able to be displaced by means of an assembly comprising a screw (8) and a nut (10) in order to allow the surgeon to slide the slide and place the knee in a state of tension.

6. Device according to claim 5, characterised in that the sliding means (3) have an internal runner in which the slide (5) is guided, and an outer surface which allows the drilling guide (30) and other components of the device to be guided, the slide having a portion which allows the drilling guide to be moved on this device.

7. Device according to claim 1, characterised in that the drilling guide (30) is constructed so as to receive a palpating arm which is capable of pressing on the anterior surface of the femoral end in order to limit the insertion of the guide on the guiding means in order to optimise the drilling position.

8. Device according to claim 1, characterised in that the slide (5) has reliefs (11) which allow precise positioning, relative to the slide, of the plate (21) of the ancillary component (20) and a rapid fixing means (6) which allows the plate (21) to be temporarily fixedly joined relative to the slide (5).

9. Device according to claim 1, characterised in that the size estimation component (40) for palpating the anterior end of the femur is formed in one piece with a member (41) which is capable of sliding on or in the sliding means (3), this sliding member having a transverse palpating arm (43) which is articulated about a shaft (44) parallel with the sliding axis.
